(19) United States
(12) Patent Application Publication
(10) Pub. No.: US 2004/0122044 A1
(43) Pub. Date: Jun. 24, 2004

(54) Utilization of substituted imidazo [1,2-A]-pyridin-3-yl-amide and imidazo [1,2-A]-pyridin-3-yl-amine compounds in pharmaceutical formulations

(76) Inventors: Corinna Maul, Aachen (DE); Hagen-Heinrich Hennies, Simmerath (DE); Bernd Sundermann, Aachen (DE)

Correspondence Address: CROWELL & MORING LLP, INTELLECTUAL PROPERTY GROUP, P.O. BOX 14300, WASHINGTON, DC, 20044-4300, US

(21) Appl. No.: 10/677,960

(22) Filed: Oct. 3, 2003

Related U.S. Application Data
(63) Continuation of application No. PCT/EP02/03796, filed on Apr. 5, 2002.

(30) Foreign Application Priority Data
Apr. 5, 2001 (DE) 101 17 184.6

(51) Int. Cl. A61K 31/4745

(52) U.S. Cl. 514/303

(57) ABSTRACT

Methods of using pharmaceutical formulations with imidazo[1,2-a]-pyridin-3-yl-amide or -amine compounds as active ingredients to inhibit nitric oxide synthase are disclosed. Treatments of certain conditions using imidazo[1,2-a]-pyridin-3-yl-amide or -amine compounds are also disclosed, as are processes for preparing imidazo[1,2-a]-pyridin-3-yl-amide or -amine compounds.

### CROSS-REFERENCE TO RELATED APPLICATIONS

This application is a continuation of International Patent Application No. PCT/EP02/03796, filed Apr. 5, 2002, designating the United States of America, and published in German as WO 02/080911, the entire disclosure of which is incorporated herein by reference. Priority is claimed based on Federal Republic of Germany Patent Application No. DE 101 17 184.6, filed Apr. 5, 2001.

### FIELD OF THE INVENTION

The present invention relates to the use of substituted imidazo[1,2-a]-pyridin-3-yl-amide and imidazo[1,2-a]-pyridin-3-yl-amine compounds and their physiologically acceptable salts as inhibitors for nitric oxide synthase and for the preparation of pharmaceutical formulations, and to a process for their preparation.

### BACKGROUND OF THE INVENTION

Nitric oxide (NO) regulates numerous physiological processes, inter alia neurotransmission, relaxation and proliferation of the smooth musculature, adhesion and aggregation of thrombocytes and tissue injury and inflammation. Because of the large number of signal functions, a connection is made between nitric oxide and a number of diseases, for example in L. J. Ignarro, Angew. Chem. (1999), 111, pages 2002-2013 and in F. Murad, Angew. Chem. Int. Ed. (1999), 111, pages 1976-1989. The enzyme responsible for the physiological formation of nitric oxide, nitric oxide synthase (NO synthase), plays an important role here in therapeutic influencing of these diseases. Three different iso-forms of NO synthase have so far been identified, that is to say the two constitutive forms nNO synthase and eNO synthase and the inducible form iNO synthase (A. J. Hobbs, A. Higgs, S. Moncada, Annu. Rev. Pharmacol. Toxicol. (1999), 39, pages 191-220; I. C. Green, P.-E. Chabrier, DDT (1999), 4, pages 47-49; P.-E. Chabrier et al., Cell. Mol. Life Sci. (1999), 55, pages 1029-1035).

The inhibition of NO synthase opens up new therapy procedures for various diseases connected with nitric oxide (A. J. Hobbs et al., Annu. Rev. Pharmacol. Toxicol. (1999), 39, pages 191-220; I. C. Green, P.-E. Chabrier, DDT (1999), 4, pages 47-49; P.-E. Chabrier et al., Cell. Mol. Life Sci. (1999), 55, pages 1029-1035), such as, for example, migraine (L. L. Thomsen, J. Olesen, Clinical Neuroscience (1998), 5, pages 28-33; L. H. Lassen et al., The Lancet (1997), 349, 401-402), septic shock, neurodegenerative diseases, such as multiple sclerosis, Parkinson's disease, Alzheimer's disease or Huntington's disease, inflammations, inflammatory pain, cerebral ischaemia, diabetes, meningitis and arteriosclerosis. The inhibition of NO synthase can moreover have an effect on wound healing, on tumours and on angiogenesis and cause a non-specific immunity to microorganisms (A. J. Hobbs et al., Annu. Rev. Pharmacol. Toxicol. (1999), 39, pages 191-220).

Active compounds known to date which inhibit NO synthase include, in addition to L-NMMA and L-NAME—i.e. analogues of L-arginine, from which nitric oxide and citrulline are formed in vivo with the participation of NO synthase—inter alia S-methyl-L-citrulline, aminoguanidine, S-methylisourea, 7-nitroindazole and 2-mercaptoethylguanidine (A. J. Hobbs et al., Annu. Rev. Pharmacol. Toxicol. (1999), 39, pages 191-220).

### SUMMARY OF THE INVENTION

One object of the present invention is to provide pharmaceutical formulations which act as an inhibitor on NO synthase. A further object is to provide pharmaceutical formulations suitable for treatment of migraine, septic shock, neurodegenerative diseases, such as multiple sclerosis, Parkinson's disease, Alzheimer's disease or Huntington's disease, inflammations, inflammatory pain, cerebral ischaemia, diabetes, meningitis, arteriosclerosis, fungal diseases or for wound healing.

Surprisingly, it has now been found that substituted imidazo[1,2-a]-pyridin-3-yl-amide and -amine compounds corresponding to formula I act as inhibitors on NO synthase and are suitable in particular for treatment of migraine, septic shock, neurodegenerative diseases, such as multiple sclerosis, Parkinson's disease, Alzheimer's disease or Huntington's disease, inflammations, inflammatory pain, cerebral ischaemia, diabetes, meningitis, arteriosclerosis, fungal diseases or for wound healing.

In one embodiment, the invention provides a method of inhibiting NO synthase in a mammal. The method comprises administering to the mammal an effective NO synthase inhibiting amount of at least one substituted imidazo[1,2-a]-pyridin-3-yl-amide or -amine compound corresponding to formula I

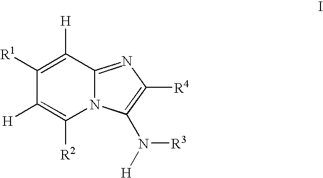

wherein,

R¹ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, F, Cl, Br, I, CN, NO₂, NH₂, C(═O)R⁵, CO₂H, CO₂R⁶, OH or OR⁷, preferably an unsubstituted or at least monosubstituted C₁₋₈-alkyl radical, F, Cl, Br, CN, NO₂, NH₂, C(═O)R⁵, CO₂H, CO₂R⁶, OH or OR⁷, particularly preferably an unsubstituted or at least monosubstituted C₁₋₈-alkyl radical,

R² represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, H, F, Cl, Br, I, CN, NO₂, NH₂, C(═O)R⁵, CO₂H, CO₂R⁶, OH or OR⁷, preferably an unsubstituted or at least monosubstituted C₁₋₈-alkyl radical or H, particularly preferably H,

R³ represents H, C(═O)R⁸ or SO₂R⁸, preferably H or C(═O)R⁸, particularly preferably H,

R⁴ represents H, an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₇-heterocyclyl radical, an unsubstituted or at least monosubstituted aryl or heteroaryl radical, a C₃₋₈-cycloalkyl radical which is bonded via a C₁₋₈-alkylene group, a C₃₋₇-heterocyclyl radical which is bonded via a C₁₋₈-alkylene group, an unsubstituted or at least monosubstituted aryl or heteroaryl radical which is bonded via a C₁₋₈-alkylene group, preferably an unsubstituted or at least monosubstituted C₁₋₈-alkyl radical or an unsubstituted or at least monosubstituted aryl or heteroaryl radical, particularly preferably an unsubstituted or at least monosubstituted aryl or heteroaryl radical,

R⁵ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, a C₃₋₇-heterocyclyl radical, an unsubstituted or at least monosubstituted aryl or heteroaryl radical or an unsubstituted or at least monosubstituted aryl or heteroaryl radical which is bonded via a C₁₋₈-alkylene group, preferably an unsubstituted or at least monosubstituted C₁₋₈-alkyl radical or an unsubstituted or at least monosubstituted aryl or heteroaryl radical,

R⁶ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical or an unsubstituted or at least monosubstituted aryl or heteroaryl radical which is bonded via a C₁₋₈-alkylene group, preferably an unsubstituted or at least monosubstituted C₁₋₈-alkyl radical or an unsubstituted or at least monosubstituted aryl or heteroaryl radical,

R⁷ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical or an unsubstituted or at least monosubstituted aryl or heteroaryl radical which is bonded via a C₁₋₈-alkylene group, preferably an unsubstituted or at least monosubstituted C₁₋₈-alkyl radical or an unsubstituted or at least monosubstituted aryl or heteroaryl radical,

R⁸ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, an unsubstituted or at least monosubstituted aryl or heteroaryl radical which is bonded via a C₁₋₈-alkylene group, preferably an unsubstituted or at least monosubstituted C₁₋₈-alkyl radical or an unsubstituted or at least monosubstituted aryl or heteroaryl radical.

Preferably said compound is in the form of its base or a physiologically acceptable salt.

Preferred C₁₋₈-alkyl radicals are selected from the group consisting of methyl, ethyl, n-propyl, 2-propyl, n-butyl, iso-butyl, sec-butyl, tert-butyl, n-pentyl, iso-pentyl, neo-pentyl, n-hexyl, 2-hexyl and n-octyl.

Preferred C₂₋₈-alkenyl radicals are selected from the group consisting of ethenyl (vinyl), propenyl (—CH₂CH═CH₂, —CH═CH—CH₃, —C(═CH₂)—CH₃), butenyl, pentenyl, hexenyl and octenyl.

Preferred C₂₋₈-alkinyl radicals are selected from the group consisting of ethinyl, propinyl (—CH—C≡CH, —C≡C—CH₃), butinyl, pentinyl, hexinyl and octinyl.

If the C₁₋₈-alkyl radical, the C₂₋₈-alkenyl radical or the C₂₋₈-alkinyl radical is present in a mono- or polysubstituted form, one or more hydrogen radical(s) is (are) preferably replaced by a substituent selected from the group consisting of F, Cl, Br, I, CN, NH₂, NH-alkyl, NH-aryl, NH-heteroaryl, NH-alkyl-aryl, NH-alkyl-heteroaryl, NH-heterocyclyl, NH-alkyl-OH, N(alkyl)₂, N(alkyl-aryl)₂, N(alkyl-heteroaryl)₂, N(heterocyclyl)₂, N(alkyl-OH)₂, NO, NO₂, SH, S-alkyl, S-aryl, S-heteroaryl, S-alkyl-aryl, S-alkyl-heteroaryl, S-heterocyclyl, S-alkyl-OH, S-alkyl-SH, OH, O-alkyl, O-aryl, O-heteroaryl, O-alkyl-aryl, O-alkyl-heteroaryl, O-heterocyclyl, O-alkyl-OH, CHO, C(═O)C₁₋₆-alkyl, C(═S)C₁₋₆-alkyl, C(═O)aryl, C(═S)aryl, C(═O)C₁₋₆-alkyl-aryl,

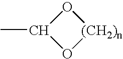

where n=1, 2 or 3, C(═S)C₁₋₆-alkyl-aryl, C(═O)-heteroaryl, C(═S)-heteroaryl, C(═O)-heterocyclyl, C(═S)-heterocyclyl, CO₂H, CO₂-alkyl, CO₂-alkyl-aryl, C(═O)NH₂, C(═O)NH-alkyl, C(═O)NH-aryl, C(═O)NH-heterocyclyl, C(═O)N(alkyl)₂, C(═O)N(alkyl-aryl)₂, C(═O)N(alkyl-heteroaryl)₂, C(═O)N(heterocyclyl)₂, SO-alkyl, SO₂-alkyl, SO₂NH₂, SO₃H, cycloalkyl, aryl, heteroaryl and heterocyclyl, wherein polysubstituted C₁₋₈-alkyl, C₂₋₈-alkenyl or C₂₋₈-alkinyl radicals are understood to mean those radicals which are poly-, e.g. di- or trisubstituted either on different atoms or on the same atom of the particular radical, for example trisubstituted on the same carbon atom, as in the case of CF₃ or —CH₂CF₃, or on different atoms, as in the case of —CH(OH)—CH═CH—CHCl₂. The polysubstitution can be by the same or by different substituents. If the substituent itself comprises an alkyl group, this is preferably selected from the group consisting of methyl, ethyl, CH₂—OH and CF₃.

The expression “C₃₋₈-cycloalkyl radical” in the context of the present invention includes cyclic hydrocarbons having 3 to 8 carbon atoms, which may be saturated or unsaturated, unsubstituted or at least monosubstituted, wherein bonding of the cycloalkyl radical to the base skeleton of formula I can be via any desired ring member of the cycloalkyl radical. The C₃₋₈-cycloalkyl radical is preferably selected from the group consisting of cyclopropyl, cyclobutyl, cyclopentyl, cyclohexyl, cycloheptyl, cyclooctyl, cyclopentenyl, cyclohexenyl, cycloheptenyl and cyclooctenyl. The C₃₋₈-cycloalkyl radical is particularly preferably a cyclohexyl radical.

The expression “C₃₋₇-heterocyclyl radical” in the context of the present invention includes a 3-, 4-, 5-, 6- or 7-membered cyclic organic radical which has at least 1, optionally also 2, 3, 4 or 5 heteroatoms in the ring system, wherein the heteroatoms can be identical or different and the cyclic radical is saturated or unsaturated but not aromatic and can be unsubstituted or at least monosubstituted. Bonding of the heterocyclyl radical to the base skeleton of the general formula I can be via any desired ring member of the heterocyclyl radical. The heterocyclyl radical can also be part of a bi- or polycyclic system. Preferred heteroatoms are selected from the group consisting of nitrogen, oxygen and sulfur. The C₃₋₇-heterocyclyl radical is preferably selected from the group consisting of tetrahydrofuryl, tetrahydropyranyl, pyrrolidinyl, piperidinyl, piperazinyl and morpholinyl.

The expression “aryl radical” in the context of the present invention denotes aromatic hydrocarbons, which can also be fused with further saturated, at least partly unsaturated or aromatic ring systems, wherein bonding of the aryl radical to the base skeleton of the general formula I can be via any desired ring member of the aryl radical. If the aryl radical has more than one substituent, these can be identical or different and can be present in any desired and possible position of the aryl radical. The aryl radical is preferably selected from the group consisting of unsubstituted or at least monosubstituted phenyl, anthracenyl, 1-naphthyl and 2-naphthyl. The aryl radical is particularly preferably selected from the group consisting of phenyl, 3-hydroxyphenyl, 3-methoxyphenyl, 2,3-dihydroxyphenyl, 2,3-dimethoxyphenyl and 1-naphthyl.

The expression “heteroaryl radical” in the context of the present invention represents a 5-, 6- or 7-membered cyclic aromatic radical which has at least 1, optionally also 2, 3, 4 or 5 heteroatoms, wherein the heteroatoms can be identical or different and wherein bonding to the base skeleton of the general formula I can be via any desired and possible ring member of the heteroaryl radical. If the heteroaryl radical has more than one substituent, these heteroaryl substituents can be identical or different and can be present in any desired and possible position on the heteroaryl ring. The heterocyclic radical can also be fused with further saturated, at least partly unsaturated or aromatic ring systems. Preferred heteroatoms are selected from the group consisting of nitrogen, oxygen and sulfur. The heteroaryl radical is preferably selected from the group consisting of unsubstituted or at least monosubstituted pyrrolyl, furyl, thienyl, pyrazolyl, imidazolyl, pyridinyl, pyridazinyl, pyrimidinyl, pyrazinyl, pyranyl, indolyl, indazolyl, purinyl, pyrimidinyl, indolizinyl, quinolinyl, isoquinolinyl, quinazolinyl, carbazolyl, phenazinyl and phenothiazinyl. Particularly preferred heteroaryl radicals are selected from the group consisting of pyridin-2-yl, pyridin-3-yl, furan-2-yl, furan-3-yl, 5-hydroxymethylene-furan-2-yl, 5-nitro-furan-2-yl, 5-[1,3]-dioxolane-furan-2-yl, 5-carboxylic acid-furan-2-yl, thien-2-yl (2-thiophene), thien-3-yl (3-thiophene) and 5-carboxylic acid-2-thiophene (5-carboxylic acid-thien-2-yl).

If the C₃₋₈-cycloalkyl, the C₃₋₇-heterocyclyl, the aryl or the heteroaryl radical is mono- or polysubstituted, this is understood to mean mono- or poly- e.g. di-, tri- or tetrasubstitution of one or more hydrogen atoms of the ring system by a substituent selected from the group consisting of F, Cl, Br, I, CN, NH₂, NH-alkyl, NH-aryl, NH-heteroaryl, NH-alkyl-aryl, NH-alkyl-heteroaryl, NH-heterocyclyl, NH-alkyl-OH, N(alkyl)₂, N(alkyl-aryl)₂, N(alkyl-heteroaryl)₂, N(heterocyclyl)₂, N(alkyl-OH)₂, NO, NO₂, SH, S-alkyl, S-cycloalkyl, S-aryl, S-heteroaryl, S-alkyl-aryl, S-alkyl-heteroaryl, S-heterocyclyl, S-alkyl-OH, S-alkyl-SH, OH, O-alkyl, O-cycloalkyl, O-aryl, O-heteroaryl, O-alkyl-aryl, O-alkyl-heteroaryl, O-heterocyclyl, O-alkyl-OH, CHO, C(═O)C₁₋₆-alkyl, C(═S)C₁₋₆-alkyl, C(═O)aryl, C(═S)aryl, C(═O)C₁₋₆-alkyl-aryl,

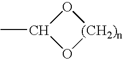

where n=1, 2 or 3, C(═S)C₁₋₆-alkyl-aryl, C(═O)-heteroaryl, C(═S)-heteroaryl, C(═O)-heterocyclyl, C(═S)-heterocyclyl, CO₂H, CO₂-alkyl, CO₂-alkyl-aryl, C(═O)NH₂, C(═O)NH-alkyl, C(═O)NHaryl, C(═O)NH-heterocyclyl, C(═O)N(alkyl)₂, C(═O)N(alkyl-aryl)₂, C(═O)N(alkyl-heteroaryl)₂, C(═O)N(heterocyclyI)₂, S(O)-alkyl, S(O)-aryl, SO₂-alkyl, SO₂-aryl, SO₂NH₂, SO₃H, CF₃, ═O, ═S; alkyl, cycloalkyl, aryl, heteroaryl and heterocyclyl, wherein a substituent can in turn be optionally substituted. Polysubstitution in this case may be by identical or different substituents. For “aryl radicals”, particularly preferred substituents are selected from the group consisting of F, CF₃, OH and O—CH₃. For “heteroaryl radicals”, particularly preferred substituents are selected from the group consisting of OH, O—CH₃, CH₂OH, NO₂, CO₂H, CO₂ethyl and [1,3]-dioxolane. For “cycloalkyl radicals”, particularly preferred substituents are CO₂H or CO₂ethyl.

The use of the compound 7-methyl-2-thiophen-3-yl-imidazo[1,2-a]pyrididin-3-yl-amine or a physiologically acceptable salt thereof, preferably the corresponding hydrochloride, as an inhibitor for NO synthase is very particularly preferred.

If the substituted imidazo[1,2-a]-pyridin-3-yl-amide and -amine compounds of formula I employed according to the invention or physiologically acceptable salts thereof have at least one asymmetric center, they can exist in the form of their racemates, their pure enantiomers, their pure diastereomers or in the form of a mixture of at least two of the abovementioned stereoisomers. The substituted imidazo[1,2-a]-pyridin-3-yl-amide and -amine compounds of formula I can also exist in the form of a mixture of their enantiomers or diastereomers. These mixtures can include two or more of the particular stereoisomers in any desired mixing ratio. Chiral substituted imidazo[1,2-a]-pyridin-3-yl-amide and -amine compounds of formula I in the enantiomerically pure form are preferably used.

The present invention also provides a process for the preparation of substituted imidazo[1,2-a]-pyridin-3-yl-amine compounds of formula I given above, wherein the radical R³ represents H and the radicals R¹, R² and R⁴ to R⁷ have the meanings given above, in which at least one substituted 2-aminopyridine corresponding to formula II

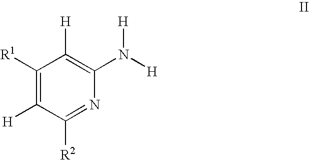

wherein the radicals R¹ and R² have the meanings given above for formula I, is reacted in solution with at least one aldehyde corresponding to formula III

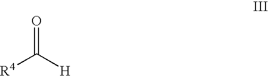

wherein the radical R⁴ has the meaning given above for formula I, and at least one alkali metal cyanide under irradiation with microwaves, and the resulting compounds of formula I in which the radical R³ represents H and the radicals R¹, R² and R⁴ to R⁷ have the meanings given above for formula I, are purified, if necessary, by conventional methods known in the art and are optionally isolated.

The power at which the irradiation with microwaves is carried out and the frequency of the irradiating microwaves can vary over a wide range.

The irradiation with microwaves is preferably carried out at a power of 100 to 1,200 watts, particularly preferably 100 to 250 watts.

The frequency of the irradiating microwaves is preferably in the range from 850 to 22,250 MHz, particularly preferably in the range of 915±25 MHz, 2,450±13 MHz, 5,800±75 MHz or 22,125±125 MHz.

The duration of the reaction for carrying out the process according to the invention can vary as a function of a large number of parameters, for example the nature of the particular compounds of formula II or III, the nature of the solvent or the reaction temperature. The particular optimum duration of the reaction can be determined by a skilled worker by simple preliminary experiments.

In a preferred embodiment of the process according to the invention, the reaction is carried out at a temperature up to a maximum of the boiling point of the solvent or solvent mixture employed. The reaction is particularly preferably carried out under reflux of the solvent or solvent mixture.

The point in time at which the irradiation with microwaves is started and the duration of the microwave irradiation can also vary.

In a preferred embodiment of the process according to the invention at least one substituted 2-aminopyridine of formula II and at least one aldehyde of formula III are first reacted with one another under irradiation with microwaves, and the resulting reaction mixture is cooled and then reacted with at least one alkali metal cyanide, optionally at elevated temperature. Alternatively, it is also possible to start the irradiation with microwaves only after addition of all the reaction components to the solvent or solvent mixture.

In a preferred embodiment of the process according to the invention, a substituted 2-aminopyridine of formula II, an aldehyde of formula III and an alkali metal cyanide are reacted with one another in equimolar amounts.

The aldehydes of formula III can be employed either in the pure form or in the form of their addition compounds, in particular in the form of their bisulfite adducts.

The alkali metal cyanide employed in the process according to the invention is preferably potassium cyanide, sodium cyanide or a mixture thereof, particularly preferably potassium cyanide.

The process according to the invention for the preparation of substituted imidazo[1,2-a]-pyridin-3-yl-amine compounds of formula I, wherein the radical R³ represents H and the radicals R¹, R² and R⁴ to R⁷ have the meanings given above, can be carried out both in nonpolar and in polar solvents, it being possible for the polar solvents to be both protic and aprotic. Mixtures of the abovementioned solvents can also be employed.

Water or a water-based solvent mixture is preferably employed as the solvent in the process according to the invention. The process according to the invention can be carried out either under normal pressure or under reduced or elevated pressure. It is preferably carried out under elevated pressure, particularly preferably under a pressure of up to 3 bar.

The substituted 2-aminopyridines of formula II and the aldehydes of formula III are generally commercially available or can be prepared by conventional methods known to persons skilled in the art.

The present invention also provides a process for the preparation of substituted imidazo[1,2-a]-pyridin-3-yl-amide compounds of formula I, wherein the radical R³ represents (C═O)R⁸ and the radicals R¹, R² and R⁴ to R⁸ have the meanings given above, in which at least one compound of formula I, wherein the radical R³ represents H and the radicals R¹, R² and R⁴ to R⁷ have the meanings given above for formula I, is reacted with at least one compound corresponding to formula R⁸—(C═O)—OH, R⁸—(C═O)—X or R⁸—(C═O)—O—(C═O)—R⁸, wherein X represents Cl, Br or I and the radical R⁸ in each case has the meaning given above for formula I, to yield a compound of formula I, wherein the radical R³ represents (C═O)R⁸ and the radicals R¹, R² and R⁴ to R⁸ have the meanings given above, and this is purified, if necessary, by conventional methods known to those skilled in the art and is optionally isolated.

The present invention also provides a process for the preparation of substituted imidazo[1,2-a]-pyridin-3-yl-amide compounds corresponding to formula I, wherein the radical R³ represents SO₂R⁸ and the radicals R¹, R² and R⁴ to R⁸ have the meanings given above for formula I, in which at least one compound of formula I, wherein the radical R³ represents H and the radicals R¹, R² and R⁴ to R⁷ have the meanings given above, is reacted with at least one compound corresponding to the formula R⁸—SO₂—OH, R⁸—SO₂—X or R⁸—SO₂—O—SO₂—R⁸, wherein X represents Cl, Br or I and the radical R⁸ in each case has the meaning given above for formula I, to yield a compound of formula I, wherein the radical R³ represents SO₂R⁸ and the radicals R¹, R² and R⁴ to R⁸ have the meanings given above, and this is purified, if necessary, by conventional methods known to persons skilled in the art and is optionally isolated.

The process according to the invention for the preparation of substituted imidazo[1,2-a]-pyridin-3-yl-amide compounds of formula I, wherein the radical R³ represents (C═O)R⁸ or SO₂R⁸ and in each case the radicals R¹, R² and R⁴ to R⁸ have the meanings given above for formula I, can be carried out either in a solvent or solvent mixture or without a solvent.

A nonpolar, a polar, protic or a polar, aprotic solvent can preferably be employed as the solvent.

The temperature can vary over a wide range. The temperature is preferably 0 to 300° C., particularly preferably 5 to 250° C.

A process for the preparation of substituted imidazo[1,2-a]-pyridin-3-yl-amide compounds of formula I, wherein the radical R³ represents (C═O)R⁸ and the radicals R¹, R² and R⁴ to R⁸ have the meanings given above, in which the reaction is carried out with an excess of the compound corresponding to the formula R⁸—(C═O)—O—(C═O)—R⁸ in an aprotic solvent at a temperature of 25 to 250° C. is particularly preferred.

A process for the preparation of substituted imidazo[1,2-a]-pyridin-3-yl-amide compounds of formula I, wherein the radical R³ represents (C═O)R⁸ and the radicals R¹, R² and R⁴ to R⁸ have the meanings given above for formula I, in which the reaction is carried out with an excess of the compound of formula R⁸—(C═O)—O—(C═O)—R⁸ in the absence of a solvent under irradiation with microwaves is also particularly preferred.

The substituted imidazo[1,2-a]-pyridin-3-yl-amine and -amide compounds can be prepared in a high yield and within short reaction times by the process according to the invention. The compounds obtained by the process according to the invention are furthermore distinguished by a high purity, so that the process according to the invention is outstandingly suitable for the preparation of substance libraries by combinatorial chemistry.

The substituted imidazo[1,2-a]-pyridin-3-yl-amide and -amine compounds of formula I according to the invention can be isolated either in the form of the free base or as a salt by the process according to the invention. The free base of the particular imidazo[1,2-a]-pyridin-3-yl-amide or -amine compound of formula I is usually obtained after its preparation by the process according to the invention described above and optionally subsequent working up by conventional methods known to persons skilled in the art. The free base, obtained in this way or formed in situ without isolation, of the particular imidazo[1,2-a]-pyridin-3-yl-amide or -amine compound of formula I can then be converted, into the corresponding physiologically acceptable salt, for example by reaction with an inorganic or organic acid, preferably with hydrochloric acid, hydrobromic acid, sulfuric acid, phosphoric acid, methanesulfonic acid, p-toluenesulfonic acid, carbonic acid, formic acid, acetic acid, oxalic acid, succinic acid, tartaric acid, mandelic acid, fumaric acid, lactic acid, citric acid, glutamic acid or aspartic acid.

Conversion of the particular imidazo[1,2-a]-pyridin-3-yl-amide or -amine compound of formula I into the corresponding hydrochloride can preferably also be achieved by adding trimethylsilyl chloride (TMSCL) to the compound of formula I, as the free base, dissolved in a suitable organic solvent, such as e.g. butan-2-one (methyl ethyl ketone).

If the substituted imidazo[1,2-a]-pyridin-3-yl-amide and -amine compounds of formula I according to the invention are obtained in the form of their racemates or other mixtures of their various enantiomers and/or diastereomers by the preparation process according to the invention, these can be separated and optionally isolated by conventional processes known to persons skilled in the art. Examples of such separation processes include chromatographic separation processes, in particular liquid chromatography processes under normal pressure or under elevated pressure, preferably MPLC and HPLC processes, and processes of fractional crystallization. In this procedure, in particular, individual enantiomers, e.g. diastereomeric salts formed by HPLC on a chiral phase or by crystallization with chiral acids, for example (+)-tartaric acid, (−)-tartaric acid or (+)-10-camphorsulfonic acid, can be separated from one another.

The present invention also relates to the use of at least one substituted imidazo[1,2-a]-pyridin-3-yl-amide or -amine compound of formula I as an inhibitor for NO synthase and/or for treatment of migraine, septic shock, neurodegenerative diseases, preferably multiple sclerosis, Parkinson's disease, Alzheimer's disease or Huntington's disease, inflammations, inflammatory pain, cerebral ischaemia, diabetes, meningitis, arteriosclerosis, or fungal diseases or for wound treatment.

The present invention also provides the use of at least one substituted imidazo[1,2-a]-pyridin-3-yl-amide or -amine compound of formula I for inhibiting migraine, septic shock, neurodegenerative diseases, preferably multiple sclerosis, Parkinson's disease, Alzheimer's disease or Huntington's disease, inflammations, inflammatory pain, cerebral ischaemia, diabetes, meningitis, arteriosclerosis, or fungal diseases or for wound treatment.

The invention also relates to pharmaceutical compositions for treating or inhibiting migraine, septic shock, neurodegenerative diseases, preferably multiple sclerosis, Parkinson's disease, Alzheimer's disease or Huntington's disease, inflammations, inflammatory pain, cerebral ischaemia, diabetes, or meningitis, arteriosclerosis, fungal diseases or for wound treatment.

The corresponding pharmaceutical formulations can exist as liquid, semi-solid or solid pharmaceutical formulation forms, for example in the form of injection solutions, drops, juices, syrups, sprays, suspensions, granules, tablets, patches, capsules, plasters, suppositories, ointments, creams, lotions, gels, emulsions, aerosols or in multiparticulate form, for example in the form of pellets or granules, and can also be administered as such.

In addition to at least one substituted imidazo[1,2-a]-pyridin-3-yl-amide or -amine compound of formula I employed according to the invention, the pharmaceutical formulations according to the invention conventionally comprise further physiologically acceptable pharmaceutical auxiliary substances, which can preferably be selected from the group consisting of conventional carriers, fillers, solvents, diluents, surface-active substances, dyestuffs, preservatives, disintegrating agents, lubricants, greasing agents, flavorings and binders known to persons skilled in the art.

The choice of the physiologically acceptable auxiliary substances and the amounts thereof to be employed depend on whether the pharmaceutical formulation is to be administered orally, subcutaneously, parenterally, intravenously, intraperitoneally, intradermally, intramuscularly, intranasally, buccally, rectally or locally, for example on infections on the skin, the mucous membranes and on the eyes. Formulations in the form of tablets, coated tablets, capsules, granules, pellets, drops, juices and syrups are preferably suitable for oral administration, and solutions, suspensions, easily reconstitutable dry formulations and sprays are suitable for parenteral, topical and inhalatory administration. Compounds of formula I according to the invention in a depot in dissolved form or in a plaster, optionally with the addition of agents which promote penetration through the skin, are suitable formulations for percutaneous administration. Formulation forms which can be used orally or percutaneously can also release the corresponding compounds of formula I in a delayed manner.

The pharmaceutical formulations according to the invention are prepared with the aid of conventional means, devices, methods and processes known to those skilled in the art, such as are described, for example, in “Remington's Pharmaceutical Sciences”, ed. A. R. Gennaro, 17th ed., Mack Publishing Company, Easton, Pa. (1985), in particular in part 8, chapter 76 to 93. The corresponding literature description is incorporated herein by reference and forms part of the disclosure.

The amount of the particular imidazo[1,2-a]-pyridin-3-yl-amide or -amine compound of formula I to be administered to the patient can vary and depends, for example, on the weight or the age of the patient and on the mode of administration, the indication and the severity of the disease. From 0.1 to 5,000 mg/kg, preferably 1 to 500 mg/kg, particularly preferably 2 to 250 mg of at least one imidazo[1,2-a]-pyridin-3-yl-amide or -amine compound of formula I per kg of body weight of the patient are conventionally administered.

### Molecular Pharmacology Studies

The assays used to determine the inhibition of nitric oxide synthase by the compounds of formula I according to the invention are described in the following text:

### Nitric Oxide Synthase (NOS) Assay

This assay allows the determination of the percentage inhibition of NO synthase by a compound of formula I according to the invention by measurement of the NO synthase activity in the presence of the compound. In this procedure, NO synthase is mixed together with radioactively labelled arginine and the particular compound of formula I under suitable conditions. After interruption of the NO formation reaction at a given point in time, the amount of unreacted arginine is determined directly or indirectly. Comparison of this amount with the amount of arginine remaining from the mixture of NO synthase and arginine without addition of a compound of formula I according to the invention and under otherwise identical conditions gives the percentage inhibition of NO synthase by the compound tested. This assay can be carried out as follows:

(a) incubation of the NO synthase with labelled arginine as the substrate in a reaction vessel,

(b) separation of the labelled arginine from the labelled citrulline which forms as the product of the enzymatic reaction at a point in time at which the concentration of citrulline is increasing,

(c) measurement of the amount of arginine separated in each case.

The separation is carried out through a filter plate membrane.

This NO synthase assay is particularly suitable for a “high throughput screening” (HTS) on microtiter plates (MTP).

### HTS NO Synthase Assay: General Procedure

In this HTS NO synthase assay, radioactive arginine is used as the substrate. The assay volume can be chosen in the range between 25 μl and 250 μl, depending on the nature of the microtiter plate (MTP). Cofactors and coenzymes are added, depending on the enzyme source used. The incubation of the batches in this microtiter plate (assay MTP) according to step (a) is carried out at room temperature for between 5 and 60 minutes, depending on the enzyme activity (units) used. At the end of the incubation (step (a)), the plate is placed in a cell harvester equipped with an MTP which has a cation exchanger membrane as the filter base (filter MTP). All the batches of the assay MTP are transferred into this filter MTP and filtered with suction over a cation exchanger filter plate, a filter paper loaded with phosphate groups. The filter MTP is then washed with buffer or water. With the aid of this procedure, the arginine substrate which remains is bonded to the cation exchanger, while the radioactive citrulline formed enzymatically is washed out quantitatively. After drying of the filter MTP and addition of scintillation liquid, the bound arginine can be counted on a scintillation counter. An NO synthase reaction which has not been inhibited is reflected in a low radioactivity. An inhibited enzyme reaction means that the radioactive arginine has not been reacted. That is to say a high radioactivity is found on the filter.

### Materials Used

Arginine, L-[2,3,4-³H]-monohydrochloride; order no. NET-1123, NEN

CaCl₂ anhydrous; order no. 2388.1000; Merck KGaA

1,4-Dithiothreitol (DTT), order no. 708984; ROCHE

Na₂EDTA dihydrate; order no. 03680; FLUKA

HEPES, order no. H-3375; SIGMA

NADPH, tetrasodium salt; order no. 1585363; ROCHE

TRIS; ORDER No. 93349; FLUKA

| | |
|---|---|
| Enzyme preparation | 50 mM Tris-HCl with 1 mM EDTA: |
| buffer: | The pH of the buffer was |
| | adjusted to 7.4 at 4° C. |
| Incubation buffer | 50 mM HEPES with 1 mM EDTA; |
| (medium): | 1.25 mM CaCl₂ and 1 mM |
| | dithiothreitol. The pH of the |
| | buffer was adjusted to 7.4 at |
| | 25° C. |
| Washing medium: | H₂O |

For purposes of clarity, EDTA in the foregoing materials list means ethylenediamine tetra-acetic acid. HEPES means 4-(2-hydroxyethyl)-1-piperazineethanesulfonic acid. NADPH means nicotinamide adenine dinucleotide phosphate. Tris means tris(hydroxymethyl)aminomethane.

### Enzyme Preparation

Rat cerebelli were used as the starting tissue. The animals were narcotized and sacrificed, the brain tissue, the cerebellum, was removed, 1 ml enzyme preparation buffer (4° C.) was added per rat cerebellum and the tissue was broken down with a Polytron homogenizer for 1 min at 6,000 rpm. Thereafter, centrifugation was carried out at 4° C. for 15 min at 20,000 g and the supernatant was then decanted and frozen in portions at −80° C. (precipitate discarded).

### Incubation Batch

96-well MTP with a “well” capacity of ≦250 μl were used

Pipetting sequence: see table 1:

TABLE 1

| Substance | Molarity i.b. | μl | *Protein i.b. |
|---|---|---|---|
| Incubat. buffer | — | 100 | — |
| Test substance | variable; | variable; | — |
| | preferably | preferably | |
| | 10⁻⁵ M | 20 μl | |
| NADPH | 0.5 mM | 20 | — |
| Enzyme | — | variable; | variable; |
| (see example 3) | | maximum | maximum |
| | | volume of | amount of |
| | | the enzyme | protein |
| | | solution = | which can be |
| | | 50 μl | employed = |
| | | | 100 μg |
| [³H]substrate | variable; | variable; | — |
| | preferably | preferably | |
| | 50 nM | 10 μl | |

| | |
|---|---|
| End volume: | max. 250 μl |

*The protein determination was carried out by the method of O. H. Lowry
et al; J. Biol. Chem. 193, 265 (1951). The corresponding literature description
is incorporated herein by reference and forms part of the disclosure.
i.b. = in the batch

When the pipetting operation had ended, a lid was laid on this MTP (assay MTP). Incubation was carried out at 25° C. (room temperature (RT)) for 5-60 min, depending on the amount and activity of the enzyme employed.

The content of the assay MTP was then transferred with the aid of a 96-well cell harvester into a 96-well cation exchanger MTP (filter MTP) and filtered with suction. A single washing with 200 ml H₂O (from a trough) followed.

The plate was then dried for 1 hour at 60° C. in a drying cabinet. The bottom side of the filter MTP was then sealed with a “back seal” from underneath. Thereafter 35 μl of scintillator were pipetted in per well. The upper side of the plate was furthermore sealed with a “top seal”. After a waiting time of 1 hour, the plate was measured on a β-counter.

In HTS operation, the incubation medium, NADPH solution and enzyme solution were combined before the start of the pipetting step, so that three separate pipettings did not have to be carried out in a time-consuming manner.

### Citrulline Assay

This assay was carried out as described by D. S. Bredt and S. H. Snyder (Proc. Natl. Acad. Sci. USA (1990), 87, 682-685). The corresponding literature description is incorporated herein by reference and forms part of the present disclosure.

The invention is explained in further detail in the following text with the aid of examples. These explanations are provided merely as examples and are not intended to, nor should they be understood to, be limiting.

### EXAMPLES

General Working Instructions I

The substituted 2-aminopyridine of formula II in water was initially introduced into a three-necked flask, an equimolar amount of the bisulfite adduct of the aldehyde of the general formula III was added, and the mixture was heated under reflux for two hours under irradiation with microwaves. The reaction mixture was cooled to a temperature of 20 to 25° C., and an equimolar amount of aqueous potassium cyanide solution was added. The reaction mixture was then stirred, first for three hours at a temperature of 20 to 25° C. and then overnight at 50° C.

For working up, the reaction mixture was first filtered. The filtrate was then extracted with methylene chloride and diethyl ether and the combined extracts were dried over sodium sulfate and concentrated. The resulting crude product was dissolved in 2-butanone, and the product was precipitated by addition of half a molar equivalent of water, followed by 1.1 equivalents of chlorotrimethylsilane and subsequent stirring overnight.

General Working Instructions II

20 equivalents of acetic anhydride were added to the primary amine prepared in accordance with general working instructions I in a Teflon vessel. The vessel was closed and treated at 800 watt in a microwave oven for five to sixty minutes, such that the temperature did not exceed 100° C. After cooling to 20 to 25° C., the reaction solution was added to ice-cold, approximately five percent potassium carbonate solution and extracted with methylene chloride. The resulting organic phase was dried over sodium sulfate and/or potassium carbonate and concentrated. The crude product obtained was purified by column chromatography over silica gel, and the corresponding hydrochloride was then precipitated in accordance with general working instructions I.

### Example 1

### 7-Methyl-2 -thiophen-3-yl-imidazo[1,2-a]pyridin-3-ylamine

2.9 g 2-amino-4-methylpyridine were initially introduced into 20 ml water, 5.0 g of the bisulfite adduct of thiophene-3-carbaldehyde were added, and the mixture was heated under reflux for two hours under irradiation with microwaves.

After cooling to a temperature of 20 to 25° C., a solution of 1.74 g potassium cyanide in water was added, and the reaction mixture was stirred first for three hours at a temperature of 20 to 25° C. and then overnight at 50° C. The solid which had precipitated out, 7-methyl-2-thiophen-3-yl-imidazo[1,2-a]pyridin-3-ylamine, was filtered out and washed thoroughly with diethyl ether. The resulting crude product (3.47 g) was dissolved in 28 ml 2-butanone, and the corresponding hydrochloride was precipitated by addition of 150 μl water followed by 2.1 ml chlorotrimethylsilane and subsequent stirring overnight. The yield of 7-methyl-2-thiophen-3-yl-imidazo[1,2-a]pyridin-3-ylamine hydrochloride was 3.77 g (corresponding to 53% of the theoretical).

Molecular Pharmacology Study

The compound prepared according to example 1 was tested in the HTS NO synthase assay, as described above. The inhibition of NO synthase (10 μM) by the compound according to the invention according to example 1 was 89%.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method of inhibiting nitric oxide synthase in a mammal, said method comprising administering to said mammal an effective nitric oxide synthase inhibiting amount of at least one substituted imidazo[1,2-a]-pyridin-3-yl-amide or -amine compound corresponding to formula I 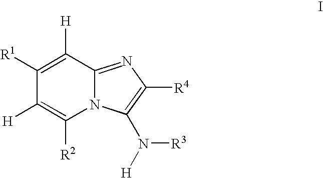
wherein,
R¹ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, F, Cl, Br, I, CN, NO₂, NH₂, C(═O)R⁵, CO₂H, CO₂R⁶, OH or OR⁷;
R² represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, H, F, Cl, Br, I, CN, NO₂, NH₂, C(═O)R⁵, CO₂H, CO₂R⁶, OH or OR⁷;
R³ represents H, C(═O)R⁸ or SO₂R⁸;
R⁴ represents H, an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₇-heterocyclyl radical, an unsubstituted or at least monosubstituted aryl or heteroaryl radical, a C₃₋₈-cycloalkyl radical which is bonded via a C₁₋₈-alkylene group, a C₃₋₇-heterocyclyl radical which is bonded via a C₁₋₈-alkylene group, or an unsubstituted or at least monosubstituted aryl or heteroaryl radical which is bonded via a C₁₋₈-alkylene group;
R⁵ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, a C₃₋₇-heterocyclyl radical, an unsubstituted or at least monosubstituted aryl or heteroaryl radical or an unsubstituted or at least monosubstituted aryl or heteroaryl radical which is bonded via a C₁₋₈-alkylene group;
R⁶ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, or an unsubstituted or at least monosubstituted aryl or heteroaryl radical which is bonded via a C₁₋₈-alkylene group;
R⁷ represents an unsubstituted or at least monosubstituted C₁₋₈-alkyl radical, an unsubstituted or at least monosubstituted C₂₋₈-alkenyl radical, an unsubstituted or at least monosubstituted C₂₋₈alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, or an unsubstituted or at least monosubstituted aryl or heteroaryl radical which is bonded via a C₁₋₈-alkylene group; and
R⁸ represents an unsubstituted or at least monosubstituted C₁₋₈-alkyl radical, an unsubstituted or at least monosubstituted C₂₋₈-alkenyl radical, an unsubstituted or at least monosubstituted C₂₋₈-alkinyl radical, a C₃₋₈-cycloalkyl radical, a C₃₋₈-cycloalkyl radical which is bonded via a C₁₋₈-alkylene group, an unsubstituted or at least monosubstituted aryl or heteroaryl radical, or an unsubstituted or at least monosubstituted aryl or heteroaryl radical which is bonded via a C₁₋₈-alkylene group;
or a salt thereof with a physiologically acceptable acid.

2. A method according to claim 1, wherein said compound is present in the form of a free base.

3. A method according to claim 1, wherein R¹ represents an unsubstituted or at least monosubstituted C₁₋₈-alkyl radical, F, Cl, Br, CN, NO₂, NH₂, C(═O)R⁵, CO₂H, CO₂R⁶, OH or OR⁷.

4. A method according to claim 1, wherein R¹ represents an unsubstituted or at least monosubstituted C₁₋₈-alkyl radical.

5. A method according to claim 1, wherein R² represents an unsubstituted or at least monosubstituted C₁₋₈-alkyl radical.

6. A method according to claim 1, wherein R² represents H.

7. A method according to claim 1, wherein R³ represents C(═O)R⁸.

8. A method according to claim 1, wherein R³ represents H.

9. A method according to claim 1, wherein R⁴ represents an unsubstituted or at least monosubstituted C₁₋₈-alkyl radical.

10. A method according to claim 1, wherein R⁴ represents an unsubstituted or at least monosubstituted aryl or heteroaryl radical.

11. A method according to claim 1, wherein R⁵ represents an unsubstituted or at least monosubstituted C₁₋₈-alkyl radical or an unsubstituted or at least monosubstituted aryl or heteroaryl radical.

12. A method according to claim 1, wherein R⁶ represents an unsubstituted or at least monosubstituted C₁₋₈-alkyl radical or an unsubstituted or at least monosubstituted aryl or heteroaryl radical.

13. A method according to claim 1, wherein, R⁷ represents an unsubstituted or at least monosubstituted C₁₋₈-alkyl radical or an unsubstituted or at least monosubstituted aryl or heteroaryl radical.

14. A method according to claim 1, wherein R⁸ represents an unsubstituted or at least monosubstituted C₁₋₈-alkyl radical or an unsubstituted or at least monosubstituted aryl or heteroaryl radical.

15. A method according to claim 1, wherein the compound of formula I is 7-methyl-2-thiophen-3-yl-imidazo[1,2-a]pyridin-3-yl-amine or a salt thereof with a physiologically acceptable acid.

16. The method of claim 1, wherein said compound is present as a hydrochloride salt.

17. A process for preparing a substituted imidazo[1,2-a]-pyridin-3-yl-amine compound corresponding to formula I according to claim 1, wherein the radical R³ represents H, comprising the step of reacting at least one substituted 2-aminopyridine corresponding to formula II 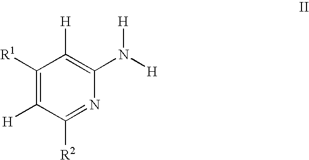
in a solvent or solvent mixture with at least one aldehyde corresponding to formula III 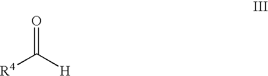
and at least one alkali metal cyanide under irradiation with microwaves, and isolating the compound of formula I wherein the radical R³ represents H.

18. A process according to claim 17, further comprising the step of purifying the compound of formula I, wherein the radical R³ represents H.

19. A process according to claim 17, wherein the irradiation with microwaves is carried out at a power of 100 to 1,200 watts.

20. A process according to claim 19, wherein the irradiation with microwaves is carried out at a power of 100 to 250 watts.

21. A process according to claim 17, wherein the irradiation is carried out with microwaves of a frequency in the range from 850 to 2,250 MHz.

22. A process according to claim 17, wherein the irradiation is carried out with microwaves of a frequency in a range selected from the group of ranges consisting of 890-940 MHz, 2,437-2463 MHz, 5,725-5875 MHz and 22,000-22,250 MHz.

23. A process according to claim 17, wherein the step of reacting is carried out at a maximum temperature of up to the boiling point of the solvent or solvent mixture.

24. A process according to claim 23, wherein the step of reacting is carried out under reflux of the solvent or solvent mixture.

25. A process according to claim 17, wherein the step of reacting comprises reacting equimolar amounts of a substituted 2-aminopyridine of formula II, an aldehyde of formula III, and an alkali metal cyanide.

26. A process according to claim 17, wherein the aldehyde of formula III is in the form of a bisulfite adduct.

27. A process according to claim 17, wherein the alkali metal cyanide is potassium cyanide, sodium cyanide or a mixture thereof.

28. A process according to claim 27, wherein the alkali metal cyanide is potassium cyanide.

29. A process according to claim 17, wherein the solvent is water or a water-based solvent mixture.

30. A process according to claim 17, wherein the step of reacting is carried out under a pressure greater than ambient pressure.

31. A process according to claim 30, wherein the step of reacting is carried out under an elevated pressure of up to 3 bar.

32. A process for preparing a substituted imidazo[1,2-a]-pyridin-3-yl-amide compound corresponding to formula I according to claim 1, wherein the radical R³ represents (C═O)R⁸, said process comprising the steps of: reacting at least one compound of formula I wherein the radical R³ represents H, with at least one compound corresponding to the formula R⁸—(C═O)—OH, R⁸—(C═O)—X or R⁸—(C═O)—O—(C═O)—R⁸ wherein X represents Cl, Br or I, to yield a compound of formula I, wherein the radical R³ represents (C═O)R⁸, and
isolating the compound of formula I wherein the radical R³ represents (C═O)R⁸.

33. The process of claim 32, further comprising the step of purifying the compound of formula I wherein the radical R³ represents (C═O)R⁸.

34. The process of claim 32, wherein the step of reacting is carried out in a nonpolar solvent or a polar, protic solvent, or a mixture thereof.

35. The process of claim 32, wherein the step of reacting is carried out in a polar, aprotic solvent or a mixture of at least two solvents selected from the group consisting of nonpolar solvents, polar, protic solvents, and polar, aprotic solvents.

36. The process of claim 32, wherein said step of reacting is carried out at a temperature of from 0 to 300° C.

37. The process of claim 36, wherein said step of reacting is carried out at a temperature of from 10 to 250° C.

38. The process of claim 32, wherein the step of reacting is carried out with an excess of the compound corresponding to the formula R⁸—(C═O)—O—(C═O)—R⁸ in an aprotic solvent at a temperature of from 25 to 250° C.

39. The process of claim 32, wherein the step of reacting is carried out with an excess of the compound corresponding to the formula R⁸—(C═O)—O—(C═O)—R⁸, without a solvent, under irradiation with microwaves.

40. A process for preparing a substituted imidazo[1,2-a]-pyridin-3-yl-amide compound corresponding to formula I according to claim 1, wherein the radical R³ represents SO₂R⁸, said process comprising the steps of reacting at least one compound of formula I wherein the radical R³ represents H with at least one compound corresponding to the formula R⁸—SO₂—OH, R⁸—SO₂—X or R⁸—SO₂—O—SO₂—R⁸ wherein X represents Cl, Br or I, to yield a compound of formula I wherein the radical R³ represents SO₂R⁸, and
isolating the compound of formula I wherein the radical R³ represents SO₂R⁸.

41. The process of claim 40, further comprising the step of purifying the compound of formula I wherein the radical R³ represents SO₂R⁸.

42. The process of claim 40, wherein the step of reacting is carried out in a nonpolar solvent or a polar, protic solvent, or a mixture thereof.

43. The process of claim 40, wherein the step of reacting is carried out in a polar, aprotic solvent or a mixture of at least two solvents selected from the group consisting of nonpolar solvents, polar, protic solvents, and polar, aprotic solvents.

44. The process of claim 40, wherein said step of reacting is carried out at a temperature of from 0 to 300° C.

45. The process of claim 44, wherein said step of reacting is carried out at a temperature of from 10 to 250° C.

46. A method for treating a condition selected from the group consisting of migraine, septic shock, neurodegenerative disease, inflammation, inflammatory pain, cerebral ischaemia, diabetes, meningitis, arteriosclerosis, fungal disease, and a wound in a mammal, said method comprising administering a pharmaceutically effective amount of a compound according claim 1 to said mammal.

47. A method according to claim 46 wherein said condition is neurodegenerative disease selected from the group consisting of multiple sclerosis, Parkinson's disease, Alzheimer's disease, and Huntington's disease.
